(19) United States
(12) Patent Application Publication
(10) Pub. No.: US 2023/0125957 A1
(43) Pub. Date: Apr. 27, 2023

(54) NON-INVASIVE THERMOMETRY SYSTEM

(71) Applicant: HYPERTHERMIA CANCER INSTITUTE LLC, Santa Monica, CA (US)

(72) Inventors: Henry Harrison LANDAU, Santa Monica, CA (US); Cynthia KEPPEL, Norfolk, VA (US)

(73) Assignee: HYPERTHERMIA CANCER INSTITUTE LLC, Santa Monica, CA (US)

(21) Appl. No.: 17/973,424

(22) Filed: Oct. 25, 2022

Related U.S. Application Data
(60) Provisional application No. 63/271,358, filed on Oct. 25, 2021.
(60) Provisional application No. 63/271,372, filed on Oct. 25, 2021.

(51) Int. Cl. G01J 5/00; G01J 5/90; G01J 5/12; A61N 7/02

(52) U.S. Cl. CPC G01J 5/0025; A61N 7/02; G01J 5/12; G01J 5/90; G01J 2005/0077

(57) ABSTRACT

A non-invasive thermometry system adapted for use during hyperthermia therapy, which has at least one infrared camera and a computer device. The infrared camera monitors temperature at the skin surface. The system may also provide depth visualization of the thermal gradient, and noninvasively monitors temperature at a tumor depth. A thermal camera, preferably an infrared camera, may be placed at a predetermined angle to an ultrasound head for a visual map of the heat signature within a patient.

### CROSS-REFERENCE TO RELATED APPLICATIONS

This application is a non-provisional of, and claims priority to, U.S. Provisional Pat. Application No. 63/271,358 filed on Oct. 25, 2021, as well as, U.S. Provisional Pat. Application No. 63/271,372 also filed on Oct. 25, 2021; which applications are incorporated herein by reference.

### STATEMENT REGARDING FEDERALLY SPONSORED-RESEARCH OR DEVELOPMENT

None.

### FIELD OF THE INVENTION

The present invention generally relates to thermometry, and more particularly relates to non-invasive thermometry systems for use during hyperthermia therapy.

### BACKGROUND

Hyperthermia therapy is the use of heat either alone or to enhance other cancer treatment methods such as chemotherapy. The temperature at which a tumor is heated is of ultimate importance, however, that needs to be mitigated by not delivering uncomfortable temperature to the patient. Optimally, hyperthermia therapy would heat only an area with a tumor. Since most tumors are sub-dermal (within a body), ultrasound has been found to be an effective method of heating the tumor region.

Using specialized crystals, focused ultrasound waves can be generated and directed to heat tumors at depth while heating surrounding areas less than many other methods. Ultrasound heats by depositing energy where the ultrasound waves are absorbed. Ultrasound, like any sound, requires energy to be created and that energy is carried with the wave. When the wave is absorbed, by the tumor for instance, the wave stops along with its energy. The energy that previously made up the ultrasound becomes thermal energy, heat, where the wave is absorbed.

The reason why ultrasound is so effective at heating only the directed area is its penetrative power. Tumors are very rarely at surface level and thus heating with no penetration would not be able to reach those tumors. While direct heat application can only directly heat the skin, ultrasound can penetrate a few centimeters below the skin to deliver its heat payload.

However, ultrasound is not perfect because most of the energy is actually absorbed before the full depth is reached, heating up the skin and other healthy tissue in the process. This is remedied using a cool water sac, or bolus, applied at the heating location. As the skin and tumor heat up, the bolus cools the skin to more comfortable temperatures by conducting heat away through the water.

One challenge with this process is the lack of thermal treatment delivery information provided to those operating the ultrasound equipment. Optimally, the hottest location will be the tumor and all adjacent locations will be below damage or discomfort temperatures. How best for a clinical operator to monitor non-invasively the temperature at the tumor depth within the body is a challenge. Current clinical thermometry used during hyperthermia treatments for cancer involves either (a) adhering individual sensors to the skin of a patient, which is an inefficient and somewhat uncomfortable process not easily duplicated between treatment sessions, or (b) utilizing sub-dermal needles at tumor depth, which is inefficient, invasive, and painful to the patient.

Measuring surface skin temperatures requires thin non-metallic probes, as metal can stop the ultrasound waves from penetrating the skin. One example is an array of thermocouples stuck to the patient directly during each treatment with sonogram gel for a measurement at the skin surface.

Measuring temperatures at tumor depth is far more difficult as few methods exist for measuring any sort of temperatures at depth in real time, especially for something as variable as the human body which has a complex internal cooling system. The currently known method for measuring temperature at a depth within the body involves thermometry needles introduced into the patient for tumor temperature measurements.

Without having the ability to reliably, and repeatedly, determine the amount of heat being delivered to a tumor at depth, clinical users of hyperthermia therapy can only guess that the heat is appropriately reaching the tumor. Combined with the desire to not burn the patient’s skin, or otherwise make the treatment intolerable, all a clinical user can do is to keep heating until the patient complains, then remove or lower the heat, and then apply again. This is uncomfortable for the patient and imprecise, at best, and ineffective at worst, for the clinical user.

Currently, while there exists systems and methods for the delivery of heat energy to a sub-dermal tumor, there is need in the art for a system and method of clinical thermometry that complements hyperthermia therapy treatments and can non-invasively provide temperature data to the clinician administering the hyperthermia therapy treatment. There is also a need in the art for a system and method that improves the efficiency, repeatability, and resolution of the thermometry while improving patient comfort during the hyperthermia therapy treatment. Additionally, the current state of the art does not address the monitoring of temperature both at the treatment head surface and at depth within the patient, in a non-invasive manner.

### SUMMARY

The invention disclosed herein includes a system, and a related method of using the system, for thermometry that is preferably adapted for and complements hyperthermia therapy treatments and non-invasively provides real-time temperature and performance data to the clinician administering the hyperthermia therapy treatment. Additionally, the invention improves the efficiency, repeatability, and resolution of the thermometry while improving patient comfort during the hyperthermia therapy treatment. Furthermore, the invention may be configured to monitor the temperature at the treatment head and at the treatment location, as well as, at depth within the patient, in a non-invasive manner.

One non-limiting embodiment of the present invention is an infrared thermometry system for non-invasively determining a temperature within a body. An exemplary embodiment includes: a processor device; at least one infrared camera operably coupled to the processor device, capable of capturing and transmitting surface temperature spatial data to the processor device; a relational database, operably coupled to the processor device, containing data correlating surface temperature data to temperature at depth within the body; and a memory operably coupled to the processor device. The memory stores computer-executable instructions that are executed by the processor.

The system is thus configured to provide an improvement over the current state of clinical thermometry in that the infrared camera(s) capture surface temperature data and dynamically transmits that data to the processor. The processor then utilizes the captured data to derive a temperature at depth and reports that information back to the clinician and/or the system for heat adjustment or enhanced cooling as needed. In this way, the system is configured to non-invasively provide temperature data at a depth within the body and to update that data dynamically to the clinical user.

It is one of the main objects of the present invention to provide a non-invasive thermometry system for use during hyperthermia therapy.

It is another object of this invention to provide an infrared thermometry system used during hyperthermia therapy, which has an infrared camera for tumor temperature monitoring.

It is another object of this invention to provide an infrared thermometry system used during hyperthermia therapy, which has an infrared camera allowing thermal depth measurements.

It is another object of this invention to provide an infrared thermometry system used during hyperthermia therapy, which has an infrared camera allowing gradient measurements.

It is another object of this invention to provide a non-invasive thermometry system used during hyperthermia therapy that may be operated without touching the patient.

It is another object of this invention to provide a non-invasive thermometry system used during hyperthermia therapy, which requires no/minimal patient set-up time.

It is another object of this invention to provide a non-invasive thermometry system used during hyperthermia therapy, which provides a full field view with high resolution.

It is another object of this invention to provide a non-invasive thermometry system used during hyperthermia therapy that can be used in conjunction with skin or other thermometry.

It is another object of this invention to provide a non-invasive thermometry apparatus used during hyperthermia therapy, which provides digitized feedback to treatment head controls and clinicians.

It is another object of this invention to provide a non-invasive thermometry apparatus used during hyperthermia therapy, which can provide thermographic data at various angles for stereoscopic or other tri-dimensional thermal image formation.

It is another object of this invention to provide a non-invasive thermometry apparatus used during hyperthermia therapy, which is of a durable and reliable construction.

Other non-limiting embodiments of the present invention are contemplated and described below and include: use of other types of sensors, other than infrared, that are configured to perform multispectral 3D scanning of the treatment area; multiple-sensor arrays configured to create a three-dimensional thermal map of the treated area; and methods of using the disclosed systems for non-invasive clinical thermometry during hyperthermia treatments.

### BRIEF DESCRIPTION OF THE DRAWINGS

In the accompanying figures, like reference numerals refer to identical or functionally similar elements throughout the separate views. The accompanying figures, together with the detailed description below are incorporated in and form part of the specification and serve to further illustrate various embodiments and to explain various principles and advantages all in accordance with the present invention, in which:

FIG. 1 is a flowchart showing a prior art method of clinical thermometry;

FIG. 2 is a schematic representation of a non-invasive thermometry system, according to an embodiment of the present invention;

FIG. 3 is an isometric representation of the non-invasive thermometry system of FIG. 2;

FIG. 4 is a flowchart representing a method of use of a non-invasive thermometry system, according to an embodiment of the present invention;

FIG. 5 is a schematic representation of a non-invasive thermometry system, according to an embodiment of the present invention; and

FIG. 6 is a schematic representation of a computing subsystem of a non-invasive thermometry system, according to an embodiment of the present invention.

### DETAILED DESCRIPTION

By way of overview and example (only), some embodiments of the present invention use infrared cameras as thermal imaging sensors in combination with a computer configured to run a programmed instruction set and interface with a relational database, in order to improve on clinical thermometry systems. In some embodiments, a technological improvement in the field of thermometry is achieved by using non-invasive thermal imaging sensors (such as infrared cameras, multispectral cameras, or three-dimensional surface temperature sensing arrays) coupled to a computing environment to dynamically compare sensed, or captured, data to stored data target models such as, for example, a target temperature at depth model. In some embodiments, an automated mechanism dynamically adjusts the configuration of hardware and/or software, to achieve desired performance objectives within a hyperthermia therapy framework. A few examples of such performance objectives include (without limitation): desired temperature at depth (tumor temperature), desired surface temperature (patient comfort metric), and treatment head temperature.

In some embodiments, dynamic monitoring/tuning allows an operator to prioritize among performance goals/objectives, such as prioritizing patient comfort over tumor temperature as well as to adjust for ambient room temperature, treatment duration, and other parameters.

Some embodiments using a non-invasive thermometry system in accordance with the present invention have at least two phases: monitoring and tuning. In some embodiments, the monitoring and tuning phases may (at least partially) overlap. Additionally, some embodiments may also have a preliminary training (calibration) phase. In a training system phase, the data can be gathered during multiple iterations of a training run. Overall system performance is monitored, as well as the progress of the training. Some examples of training parameters are: ambient room temperature, tumor location, tumor depth, body temperature, and skin initial temperature at treatment location. This can also be considered to be a “calibration” phase.

During a monitoring phase, performance can be monitored and performance data gathered, in a background process. In some embodiments, performance data can be collected from sensors that provide data on system performance with respect to a specified performance goal e.g., treatment head temperature, patient surface temperature, and ambient (room) temperature.

During a tuning phase, adjustments can be made in the area of the hyperthermia treatment by dynamically adjusting the treatment head temperature. Such tuning parameters are not the same as the training (or model calibration) parameters. For example, consider that ambient room temperature can be a training (or monitoring) parameter, while the surface (skin) temperature dynamically measured during treatment would be a tuning parameter. The desired surface (skin) temperature value of computation could be varied (or tuned) to account for the ambient temperature. Similarly, the treatment head output temperature could be varied to increase or decrease the heat delivered, with a concomitant impact on the surface (skin) temperature and, ultimately, the temperature at depth (at the tumor).

Also within the hyperthermia treatment framework, tuning parameters can be adjusted, such as the treatment head heat output. For example, adjustments can include: using the gathered treatment head and surface temperature data, both analyzed and compared in view of the training parameters and compared to the tumor target temperatures at depth, then dynamically adjusting the treatment head output temperature to meet treatment and comfort goals. Many other examples can be contemplated, within the spirit and scope of the invention.

By taking advantage of system architecture and/or system hardware and software features (e.g., spatial modeling, spectral analysis, 3D imaging, etc.), together with supports from system hardware/system software, clinical operators can dynamically monitor and adjust the tumor temperature and patient comfort goals and thereby optimize the hyperthermia treatment process.

Referring now to the drawings, a preferred embodiment of the present invention is a non-invasive thermometry system, used during hyperthermia therapy, and is generally referred to with numeral 10. It can be observed that it basically includes infrared (IR) camera 50, and computer device 60.

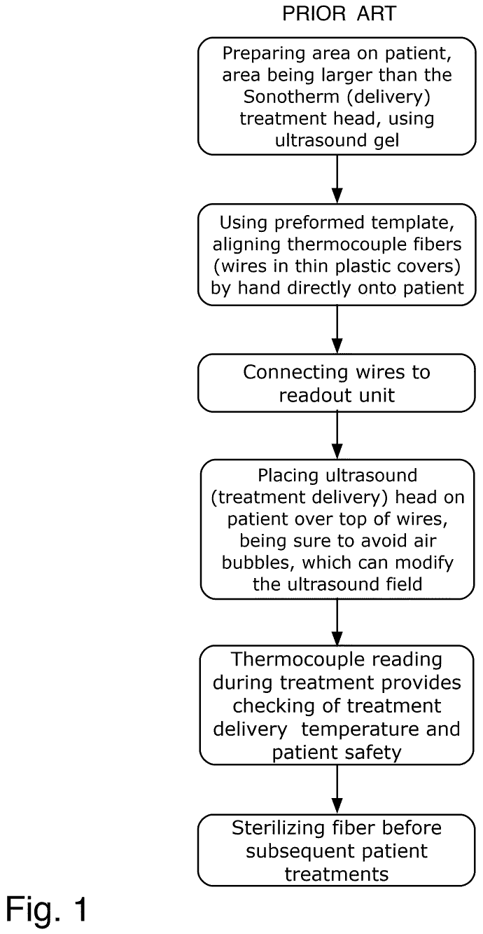

As seen in FIG. 1, according to prior art, a current method for hyperthermia treatment comprises the following steps:
preparing an area on patient P using ultrasound gel;
using a preformed template, aligning wires or thermocouple fibers 26 by hand directly onto patient P;
connecting wires to readout unit;
placing ultrasound head 40 on patient P over top of wires or fibers 26, being sure to avoid air bubbles, which can modify the ultrasound field;
reading thermocouple during treatment, which provides checking of treatment delivery temperature and patient P safety; and
sterilizing fibers 26 before subsequent patient treatments.

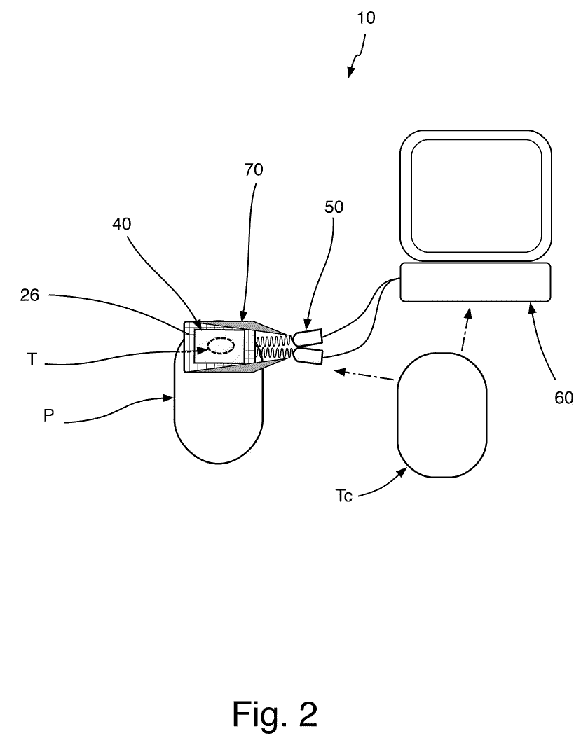

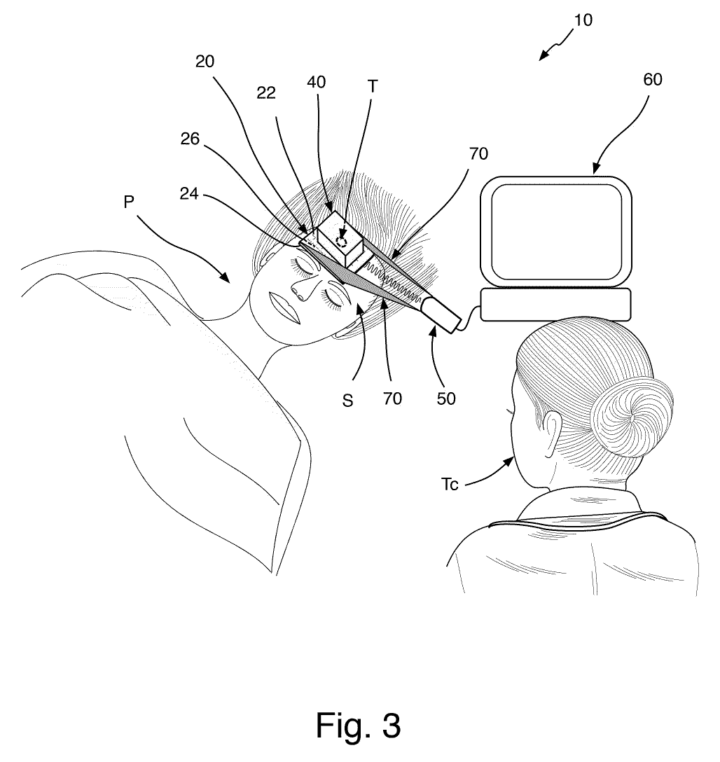

As seen in FIGS. 2 and 3, system 10 comprises at least one infrared (IR) camera 50 for clinical thermometry during hyperthermia treatment. System 10 allows for the monitoring of temperature of skin S surface along the side of a treatment head and also allows for temperature depth and gradient visualization looking at the angle to the treatment, and non-invasively monitors temperature at tumor T depth. In a preferred embodiment, the treatment head is ultrasound head 40. System 10 also works with other non-invasive and penetrating devices that deliver heat energy at a depth, such as but not limited to, microwaves and others.

In addition, utilizing two or more IR cameras 50 or moving one IR camera 50 at different angles, such as, but not limited to, two or more angles, stereographic images may be obtained, compiled, or generated.

A sensor, specifically IR camera 50, is placed at a predetermined angle to ultrasound head 40 for a visual map of a heat signature within patient P. IR camera 50 is placed at a predetermined angle to ultrasound head 40 due to the fact that a line of sight is needed when utilizing this type of sensor device.

In a preferred embodiment, system 10 comprises first and second IR Cameras 50. A first IR Camera 50 points at the object in question and centers the object within a view of IR camera 10. A second IR camera 50 is secured between about 2 to 20 inches to one side of first IR camera 50, centering the image within a field of view of second IR camera 50.

In a preferred embodiment, at least one IR camera 50 is placed at an angle perpendicular, or in a range between about 30 – 120 degrees, to treatment head or ultrasound head 40 and focused towards the treatment field. A sensor array, whether the currently-deployed fibers 26 or mat 20 or the approach is used to measure the temperature at skin S. IR camera 50 is used to monitor the thermal gradient from skin S down to tumor T, to visualize the extent and depth of the thermal field, and to verify tumor T treatment.

Ultrasound head 40 side view may be included in IR camera 50 field to calibrate IR camera 50 color map, or it may be blocked by a thermally opaque curtain 70, so that the thermal gradient within patient P is more apparent.

In a preferred embodiment, IR cameras 50 are connected to computer device 60 for live viewing. Computer device 60 may also comprise software that allows displaying the two images side by side, with some separation.

In another embodiment, a set of stereographic viewing lenses can be placed between about 1 and 2 feet away from a screen of computer device 60, therefore technician Tc, monitoring temperatures, will quickly and easily see a stereographic IR image.

The software allows adjustment of the temperature scale and selection of specific points to monitor to have the side-by-side format enabling stereographic viewing.

In another embodiment, IR camera 50 also creates 3D or pseudo-3D temperature imaging.

The depth and size of the treatment field and any thermal changes during treatment can be visualized and compared to the planned treatment, as can temperature gradients or changes therein. Moreover, the temperature along the side of treatment head 40 can be measured with precision in the same IR camera 50 capture.

IR camera 50 for tumor T monitoring comprises the following benefits:
is fully non-invasive;
requires no touching of patient P;
requires no/minimal patient P set-up time;
provides a full field view with high resolution;
provides gradient information with high resolution;
need not be sterilized between patient uses;
can be used in conjunction with, and/or normalized to, skin S or other thermometry.

IR camera 50 detects and measures the infrared energy of objects. IR camera 50 converts that infrared data into a visual and/or electronic image that shows the apparent surface temperature of the object being measured.

The technology of system 10 can be extended to other applications where non-invasive thermometry is desired. Embodiments of system 10 may be applied, for example, in temperature monitoring during surgery to monitor at-risk organs that can be getting cold from reduced blood flow, or as an ancillary indicator for breast and other cancers that cause increased or disproportionate blood supply in tumor region, and therefore increased heat flow to that region, as well as other applications.

In another embodiment, the system 10 further includes fibers 26.

In yet another embodiment, the system 10 further is used in conjunction with mat 20. Mat 20 is a thermometry mat having embedded wires or fibers 26 to provide thermal information based on the thermal coefficient of resistance (TCR) of said wires or fibers 26.

Mat 20 is placed between skin S and ultrasound head 40. Mat 20 is flexible enough to conform to the patient’s body shape at the treatment point. Mat 20 may be used with ultrasound gel to fully avoid air bubbles in the treatment field.

In embodiments, Mat 20 comprises top face 22 and bottom face 24. Between top face 22 and bottom face 24 are embedded wires or fibers 26. In a preferred embodiment, fibers 26 comprise nickel wires and other metal wires, whereby thermal sensing is based on the thermal coefficient of resistivity (TCR) of wires 26. Mat 20 is configured to be approximately the size of treatment head 40 for alignment purposes.

Mat 20 and infrared camera 50 improve repeatability and comfort of patient P while reducing room time per patient P, but the IR setups should additionally provide temperature measurements, in 3D per some embodiments, of treatment field size, shape, and gradient. System 10 also has improved resolution over current individually placed sensors.

System 10 for clinical thermometry during hyperthermia treatments also improves the patient P experience, increases the quality of treatment, and allows the clinic to treat more individuals by increasing the efficiency of each treatment session

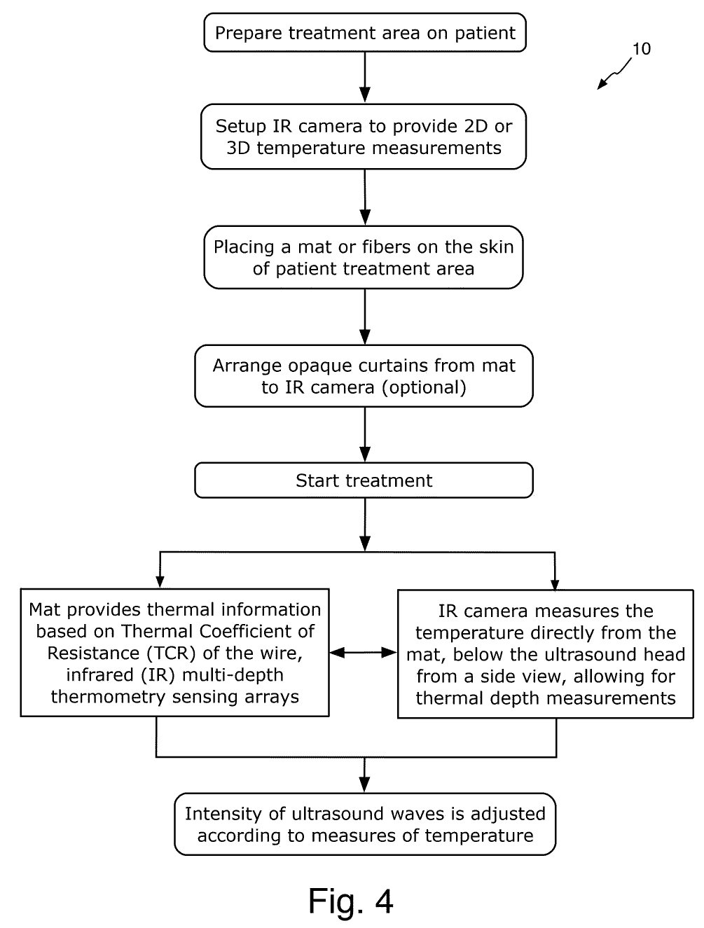

As seen in FIG. 4, a method 10 of using the non-invasive thermometry system used during hyperthermia therapy according to and embodiment comprises the following steps:
a) preparing a treatment area on patient P;
b) setting up IR camera 50 to provide 2D or 3D temperature measurements;
c) placing fibers 26 or mat 20 on patient P treatment area;
d) arranging curtains 70 from mat 20 to IR camera 50;
e) placing ultrasound head 40 over fibers 26 or mat 20; and
f) starting treatment taking mat 20 or fibers 26 temperatures, measured by IR camera 50, and adjusting ultrasound wave intensity according to the temperature field size and shape measurements.

Step d) of arranging curtains 70 from mat 20 to IR camera 50 is optional.

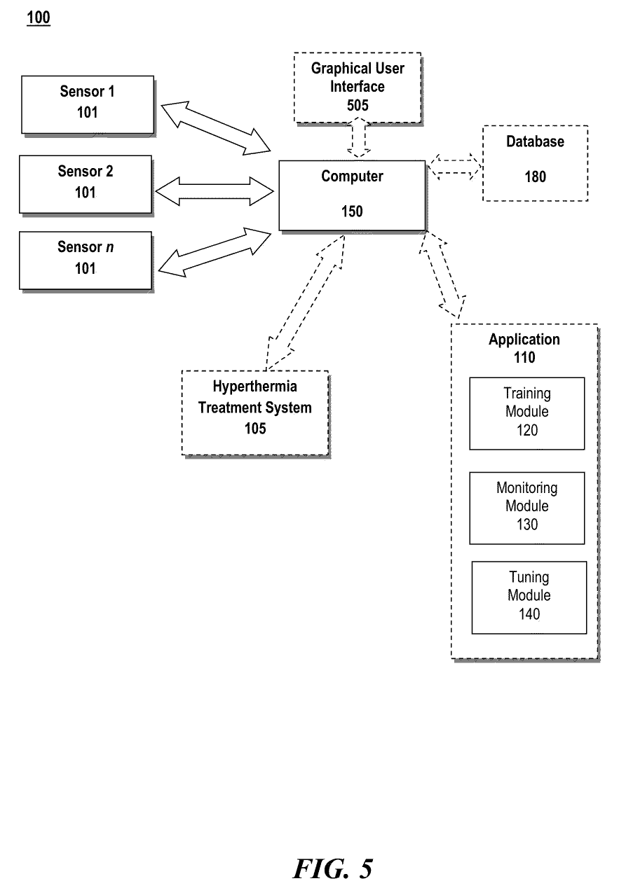

FIG. 5 is a block diagram of exemplary components of a non-invasive thermometry system 100 for use in hyperthermia treatments, according to embodiments of the present invention. As depicted, the system 100 includes a one or more sensor 101, a computer 150, and a database 180. In some embodiments, the computer 150 is integrated with one or more components of a hyperthermia treatment system 105, such as, but not limited to, an ultrasound machine with a treatment head. The computer 150 monitors and dynamically tunes the configuration of the system 100 to achieve a specified performance goal/objective (for example: treatment time, treatment temperature, patient comfort).

The computer 150 can provide a multi-phase service. In one phase, the computer 150 can work in a background process, monitoring the performance and inputs (for example: sensor data) of the system 100, while the system 100 is running in a parallel foreground process (for example, sensors collecting data). In another phase, the computer 150 dynamically adjusts the system 100 configuration based on what it has observed from the monitoring phase.

In some embodiments, the system 100 runs an application 110. It will be understood that the application (computer-executable instruction set 110) depicted here is representative of exemplary processes for machine learning and in actuality may encompass several applications, functions, algorithms, and the like, residing on a single machine or distributed across multiple machines. For compactness of disclosure, we discuss a training module 120, a monitoring module 130 and a tuning module 140. Each of these modules perform certain functions and may be carried out as software, hardware, or on a combination of software and hardware.

The training module 120 uses system software and hardware configured to support a machine learning process. For example, training module 120 takes training data (ambient room temperature, sensor quantity, sensor type(s), sensor location(s), treatment head information, etc...) and treatment parameters (treatment location, depth, target temperature at depth, target time of exposure at temperature at depth patient comfort criteria, etc...), and outputs target parameters.

Target parameters are calculated by computer 150 comparing treatment parameters in view of training data and further compared to empirical data from the database.

In embodiments, the database 180 is part of the system 100. In a system for hyperthermia treatment of tumors in cancer patients, for example, there is a tumor targeted for heat treatment and that tumor has been determined to be located at a particular depth and in a particular location of the patient’s body. Depending on the depth, size and location of the tumor, the various tissues, bone, organs, and other body structures present between the tumor and the outside surface of the skin in the area located nearest to the tumor will have a different thermal coefficient. For each different location, there is a thermal coefficient or other heat-transfer variable (multiplier) associated with the location -- these are some of the values that are stored in the database 180. The heat-transfer variables are derived from experimental calculations, prior known probe measurements, or other empirical data sources. During the training phase, the computer 150 queries the database and pulls values related to the treatment parameters as modified by the training data. The computer 150 derives target parameters from this machine learning process. These target parameters are fed into the monitoring module 130 for use in a monitoring phase.

The actions taken by the computer 150 can be very different, depending on the desired performance objective. The desired performance objective is achieved by observing and monitoring the performance of the system 100 and dynamically fine-tuning the system 100.

During the monitoring phase, the computer 150 runs a monitoring module 130 using system software and hardware configured to support a machine learning process. Real-time data captured from the sensors is used by the monitoring module and compared to the target parameters from the training module. Logical comparators applied to the input data by the monitoring module calculate deviations of the actual (real-time) sensor data from the target parameters. If the comparison yields a deviation that is outside of a pre-set acceptable tolerance, then the monitor module sends an instruction to the tuning module to initiate a tuning phase.

During the tuning phase, adjustments can be made to the tuning parameters, such as heat output at the treatment head, or treatment duration, for example. In some embodiments, the adjustments indicated by the tuning module may be reported to a user, for example, using a user interface. In other embodiments, the adjustments indicated by the tuning module may be accomplished by the system 100 interfacing directly with the tunable apparatus (for example, a sonogram machine), hyperthermia treatment system 105, in order to automatically adjust the tuning parameters without any direct human interaction.

The system 100 can contain a graphical user interface (GUI) 505 that allows a user to select and view a specific performance objective. Each performance goal is related to measurable performance criteria. The performance objective can be changed in real-time, as desired by the operator. Performance objectives may need to be changed in response to environmental changes, changes in input data, or for other reasons. The system monitoring and tuning is performed according to the current performance objective.

In some embodiments, once the system 100 identifies that performance is straying from the pre-selected performance objective during the monitoring phase, the computer 150 attempts to identify whether changing any of the performance parameter values will bring the system closer to the performance objective. If such values exist, the computer 150 will identify a performance parameter (tuning parameter) to be adjusted (either optimally or not) and instruct the system 100 to use the new parameter value. This automatic identification and selection of the tuning parameter can be reflected on the GUI 505.

The foregoing discloses exemplary systems for non-invasive thermometry systems, according to embodiments of the present invention. The system 100 is only one example of a suitable system and is not intended to limit the scope of use or functionality of embodiments of the present invention described above. The system 100 is operational with numerous other general purpose or special purpose computing system environments or configurations. Examples of well-known computing systems, environments, and/or configurations that may be suitable for use with the information processing system 100 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, clusters, and distributed cloud computing environments that include any of the above systems or devices, and the like.

The system 100 may be described in the general context of computer-executable instructions, being executed by a computer system. The system 100 may be practiced in various computing environments such as conventional and distributed computing environments where tasks are performed by remote processing devices that are linked through a communications network. In a distributed computing environment, program modules may be located in both local and remote computer system storage media including memory storage devices.

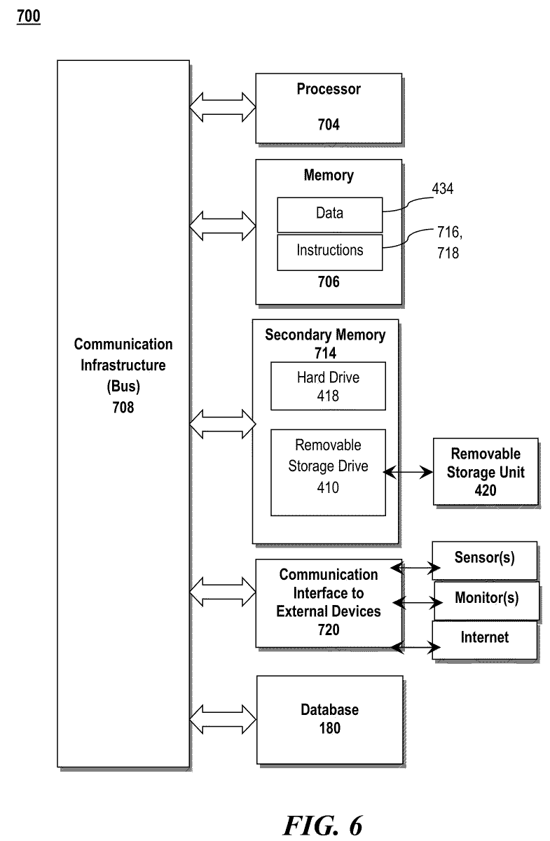

Referring to the figures in general, and FIG. 5 and FIG. 6, in particular, the computer-executable instructions are configured to define an expected target temperature spatial map at depth within the body correlated to the measured surface temperature data profile by querying the relational database. Performance objectives are set by the computer-executable instructions that identifies one or more performance criteria. A performance objective may be, for example, tumor depth versus surface temperature. Performance criteria may be, for example, desired tumor temperature at depth, comfortable patient surface temp, or other desired criteria. Through the computer-executable instructions, the processor collects and monitors the surface temperature gradient data and compares the measured surface temperature to the performance objective(s). Finally, the computer-executable instruction set causes the processor to dynamically report one or more collected, processed, or compared thermometry information to the user.

Referring again to FIG. 7, system 700 includes the computer 150. In some embodiments, computer 150 can be embodied as a general-purpose computing device. The components of computer 150 can include, but are not limited to, one or more processor devices or processing units 704, a system memory 706, and a bus 708 that couples various system components including the system memory 706 to the processor 704.

The bus 708 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

The system memory 706 can also include computer system readable media in the form of volatile memory, such as random access memory (RAM) and/or cache memory. The computer 150 can further include other removable/non-removable, volatile/non-volatile computer system storage media. By way of example only, a storage system 714 can be provided for reading from and writing to a non-removable or removable, non-volatile media such as one or more solid state disks and/or magnetic media (typically called a “hard drive”). A magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to the bus 708 by one or more data media interfaces. The memory 706 can include at least one program product embodying a set of program modules 718 that are configured to carry out one or more features and/or functions of the present invention e.g., described with reference to various FIGS. 2 - 6. Referring again to FIG. 7, program/utility 716, having a set of program modules 718, may be stored in memory 706 by way of example, and not limitation, as well as an operating system, one or more application programs, other program modules, and program data. Generally, program modules may include routines, programs, objects, components, logic, data structures, and so on that perform particular tasks or implement particular abstract data types. Each of the operating system, one or more application programs, other program modules, and program data or some combination thereof, may include an implementation of a networking environment. In some embodiments, program modules 718 are configured to carry out one or more functions and/or methodologies of embodiments of the present invention.

The computer 150 can also communicate with one or more external devices 720 that enable interaction with the computer 150; and/or any devices (e.g., network card, modem, etc.) that enable communication with one or more other computing devices. A few (non-limiting) examples of such devices include: a keyboard, a pointing device, a display presenting a graphical user interface, etc.; one or more devices that enable a user to interact with the computer 150; and/or any devices (e.g., network card, modem, etc.) that enable the computer 150 to communicate with one or more other computing devices. Such communication can occur via I/O interfaces. In some embodiments, the computer 150 can communicate with one or more networks such as a local area network (LAN), a general wide area network (WAN), and/or a public network (e.g., the Internet) via a network adapter, enabling the system 700 to access a database 180. Other hardware and/or software components can also be used in conjunction with the computer 150. Examples include, but are not limited to: microcode, device drivers, redundant processing units, external disk drive arrays, RAID systems, tape drives, and data archival storage systems.

As will be appreciated by one skilled in the art, aspects of the present invention may be embodied as a system, method, or computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Accordingly, aspects of the present invention may take the form of an entirely hardware embodiment, an entirely software embodiment (including firmware, resident software, microcode, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, although not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, although not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, or an interpreted, interactive, object-oriented programming language such as Python, or procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention have been discussed above with reference to flowchart illustrations and/or block diagrams of methods, apparatus (systems) and computer program products according to various embodiments of the invention. It will be understood that each block of the flowchart illustrations and/or block diagrams, and combinations of blocks in the flowchart illustrations and/or block diagrams, can be implemented by computer program instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer program instructions may also be stored in a non-transitory computer readable storage medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, although do not preclude the presence or addition of one or more other features, integers, steps, operations, elements, components, and/or groups thereof.

The description of the present application has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand various embodiments of the present invention, with various modifications as are suited to the particular use contemplated.

What is claimed is:

1. A non-invasive thermometry system adapted for use in hyperthermia treatments, the non-invasive thermometry system comprising:
a processor device;
at least one sensor device operably coupled to the processor device capable of capturing and transmitting surface temperature spatial data to the processor device; and
a memory operably coupled to the processor device and storing computer-executable instructions causing:
defining an expected target temperature spatial map at depth correlated to a measured surface temperature captured by the at least one sensor device by querying a relational database;
setting a performance objective that identifies one or more performance criteria;
collecting and monitoring the surface temperature gradient data;
comparing the measured surface temperatures to the performance criteria; and
dynamically reporting the comparing.

2. The non-invasive thermometry system of claim 1 where the at least one sensor device is chosen from the group consisting essentially of: infrared camera; multi-spectral imaging sensor; thermal contact sensor; thermocouple; temperature-sensing mat; and multi-layer thermometry apparatus.

3. The non-invasive thermometry system of claim 1 further comprising at least two sensor devices where a first sensor device is configured to capture data substantially at a treatment location and a second sensor device is configured to capture data substantially orthogonally to the treatment location.

4. The non-invasive thermometry system of claim 3 further comprising a three-dimensional graphical representation by the processor of the captured sensor data compiled from the at least two sensor devices.

5. The non-invasive thermometry system of claim 3 further comprising a representation by the processor of heated volume depth and geometry below treatment location assessed from the captured sensor data compiled from the at least two sensor devices.

6. The non-invasive thermometry system of claim 1 further configured to interface with a treatment head, wherein the dynamically reporting the comparing further comprises sending an instruction to modify a treatment head performance value of the treatment head.

7. The non-invasive thermometry system of claim 1 where the at least one sensor is operably connected to the processor device via a wireless connection.

8. A computer-implemented method adapted for use in a non-invasive thermometry system having an at least one tuning parameter, the computer-implemented method comprising:
providing a training module run by a computer within the non-invasive thermometry system;
providing a monitoring module run by the computer;
providing a tuning module run by the computer;
receiving, by the training module, one or more training data;
receiving, by the training module, one or more treatment parameter;
defining, by the training module, one or more target parameter;
defining, by the training module, one or more tuning parameter;
reporting, by the training module to the monitoring module, the one or more target parameter;
reporting, by the training module to the tuning module, the one or more tuning parameter;
collecting and monitoring, by the monitoring module, performance data of the non-invasive thermometry system performance;
dynamically comparing, by the monitoring module, the performance data to the one or more target parameter;
dynamically reporting, by the monitoring module to the tuning module, the results of the comparing; and
dynamically updating, by the tuning module, the non-invasive thermometry system by adjusting the at least one tuning parameter, in response to the comparing.

9. The computer-implemented method of claim 8 wherein the training module defines the one or more target parameters by comparing the one or more treatment parameter in view of the training data and further in view of empirical data from a database.

10. The computer-implemented method of claim 8 wherein the collecting and monitoring are performed in a background process.

11. The computer-implemented method of claim 8 wherein the at least one tuning parameter is selected from a group consisting of: treatment temperature delivered and treatment duration.

12. The computer-implemented method of claim 8 further comprising providing a graphical user interface with adjustable graphical elements representing real-time values of the performance data.

13. The computer implemented method of claim 8 wherein at least one of the target parameters comprises a calculated temperature at depth.

14. The computer implemented method of claim 13 further comprising providing a graphical user interface representing real-time values of the target parameter comprising a calculated temperature at depth.

15. The computer implemented method of claim 12 further comprising providing a three-dimensional graphical representation of the real-time values of the performance data.

16. A non-invasive thermometry system for use in hyperthermia treatments comprising:
a one or more sensor;
a computer configured to monitor and dynamically tune the configuration of the system to achieve a specified performance objective; and
a database.

17. The non-invasive system of claim 16 where the computer is integrated with one or more components of a hyperthermia treatment system.

18. The non-invasive system of claim 17 where the computer is further configured to monitor and dynamically tune the configuration of the hyperthermia treatment system to achieve a specified performance objective.

19. The non-invasive thermometry system of claim 16 where the computer is further configured to receive data from the one or more sensor, and compute a three-dimensional data array from the received data.

20. The non-invasive thermometry system of claim 19 further comprising a graphical interface configured to display a graphical representation of the computed three-dimensional data array.
